(19) United States
(12) Patent Application Publication
(10) Pub. No.: US 2011/0011307 A1
(43) Pub. Date: Jan. 20, 2011

(54) Method of preparing modified colored pigments

(76) Inventors: Alexander I. Shakhnovich, Westford, MA (US); Yuan Yu, Westford, MA (US); Friedrich K. von Gottberg, Cambridge, MA (US)

Correspondence Address: Cabot Corporation/FR, Law Department, 157 Concord Road, Billerica, MA, 01821-7001, US

(21) Appl. No.: 12/804,444

(22) Filed: Jul. 21, 2010

Related U.S. Application Data
(63) Continuation of application No. PCT/US2009/000432, filed on Jan. 23, 2009.
(60) Provisional application No. 61/062,310, filed on Jan. 25, 2008.

(51) Int. Cl. C09B 29/01; C09B 67/50; C09D 11/02; C07C 245/12

(52) U.S. Cl. 106/31.78; 106/31.8; 106/412; 106/496; 534/560; 534/563; 534/565

(57) ABSTRACT

The present invention relates to a method of preparing a modified colored pigment comprising a pigment having attached at least one organic group. The method comprises the step of combining, in any order, a) a solution of an organic colorant in a solvent, b) at least one aromatic amine comprising the organic group, c) at least one diazotizing agent, d) an aqueous medium, e) optionally at least one acid, and f), optionally at least one second solvent, to form the modified colored pigment.

### BACKGROUND OF THE INVENTION

1. Field of the Invention

The present invention relates to a method of preparing a modified colored pigment comprising a pigment having attached at least one organic group.

2. Description of the Related Art

A variety of procedures have been described for modifying colorants, such as dyes, carbonaceous materials, and organic colored pigments. Typically these methods involve the preparation of a dispersion of the colorant in a medium and the reaction of the surface of this colorant with various types of reagents. The resulting product is a modified colorant comprising the colorant having attached at least one organic group, such as an organic group comprising at least one ionic group, ionizable group, or a mixture thereof. Modifying colorants in this way has been shown to produce materials having desirable overall properties.

For example, modified pigments can be prepared using the methods described in U.S. Pat. Nos. 5,554,739, 5,707,432, 5,837,045, 5,851,280, 5,885,335, 5,895,522, 5,900,029, 5,922,118, and 6,042,643, and PCT Publication WO 99/23174. Such methods provide for a more stable attachment of the groups onto the pigment compared to dispersant type methods, which use, for example, polymers and/or surfactants. Other methods for preparing modified pigments include reacting a pigment having available functional groups with a reagent comprising the organic group, such as is described in, for example, U.S. Pat. No. 6,723,783. Such functional pigments may be prepared using the methods described in the references above. In addition modified carbon blacks containing attached functional groups may also be prepared by the methods described in U.S. Pat. Nos. 6,831,194 and 6,660,075, U.S. Patent Publication Nos. 2003-0101901 and 2001-0036994, Canadian Patent No. 2,351,162, European Patent No. 1 394 221, and PCT Publication No. WO 04/63289, as well as in N. Tsubokawa, Polym. Sci., 17, 417, 1992.

For organic colored pigments, it is known in the art that these colorants are typically prepared by reaction in a solvent to form the organic colorant as a solution in the solvent. The colorant is then removed from the solvent, such as by precipitation, and sometimes also undergoes further processing steps, such as milling. Since the organic colorant is prepared in a solvent, it would be desirable to modify this colorant while in solution, prior to isolation of the pigment such as by precipitation. This would eliminate the need to redisperse the pigment prior to modification, thereby providing a more efficient overall process.

### SUMMARY OF THE INVENTION

The present invention relates to a method of preparing a modified colored pigment comprising a pigment having attached at least one organic group. The method comprises the step of combining, in any order, a) a solution of an organic colorant in a solvent, b) at least one aromatic amine comprising the organic group, c) at least one diazotizing agent, d) an aqueous medium, e) optionally at least one acid, and f), optionally at least one second solvent, to form the modified colored pigment. Various embodiments of the method are described, including wherein the organic group comprises at least one ionic group, at least one ionizable group, or a mixture thereof. Also, the method of the present invention may further comprise a particle size adjustment step, either prior to or after forming the modified colored pigment.

It is to be understood that both the foregoing general description and the following detailed description are exemplary and explanatory only and are intended to provide further explanation of the present invention, as claimed.

### DETAILED DESCRIPTION OF THE INVENTION

The present invention relates to a method of preparing a modified colored pigment using a solution of an organic colorant in a solvent.

The method of the present invention comprises the step of combining a solution of an organic colorant in a first solvent, at least one aromatic amine, at least one diazotizing agent, an aqueous medium, and, optionally, an acid and/or a second solvent. These components may be combined in any order. For example, the aromatic amine, diazotizing agent, and optional acid may be combined, with or without the second solvent, to form a diazonium reagent, which is then combined with the solution of the organic colorant in the first solvent to form a mixture. The mixture, which can be either in the form of a solution or a dispersion, depending of the types and relative amounts of the solvents, discussed in more detail below, can then be combined with the aqueous medium, forming the modified colored pigment. Alternatively, the aromatic amine, diazotizing agent, optional acid, and the aqueous medium, with or without the second solvent, may be combined to form an aqueous solution of a diazonium reagent. This aqueous solution can then be combined with the solution of the organic colorant in the first solvent, thereby forming the modified colored pigment. Other combinations are also possible.

A variety of different organic colorants can be used in the method of the present invention. As used herein, an organic colorant is a chromophoric material that is a primary constituent of a pigment. It is soluble in a solvent but forms a pigment when combined with an aqueous medium, such as water. The organic colorant may be a blue colorant, a black colorant, a brown colorant, a cyan colorant, a green colorant, a white colorant, a violet colorant, a magenta colorant, a red colorant, an orange colorant, a yellow colorant, or mixtures thereof. Suitable examples of organic colorants include anthraquinones, phthalocyanine blues, phthalocyanine greens, disazos, monoazos, pyranthrones, perylenes, heterocyclic yellows, quinolonoquinolones, isoindolones, indanthrones, quinacridones, and (thio)indigoids. In particular, the organic colorant is a non-azo colorant, due to the presence of reactive materials that may interfere with the other components used in the present method, which are described in more detail below.

The organic colorant is a solution in a first solvent, and a variety of different solvents may be used as the first solvent, depending, for example, on the type of organic colorant, as long as the solvent is capable of dissolving the colorant. In addition, since the organic colorant used in the method of the present invention forms a pigment when combined with an aqueous medium; the first solvent for the organic colorant solution is preferably a non-aqueous solvent, which is a solvent that comprises less than 50% by weight water. For example, the first solvent may comprise less than 30% by weight water, including less that 20%, less than 10%, or less than 5% by weight water. The first solvent may also be anhydrous. Examples of suitable first solvents include N-methylpyrrolidone (NMP), N,N-dimethylformamide (DMF), dimethyl sulfoxide (DMSO), tetramethylene sulfone (sulfolane), chloroform, aromatic solvents (such as benzene or toluene), or hydrocarbon solvents (such as hexane, octane, or cyclohexane) as well as strong acids, such as polyphosphoric acid or sulfuric acid. While the first solvent may be water miscible, it is preferably still a non-aqueous solvent. Specific solvents for specific organic colorants will be known to one skilled in the art. For example, when the organic colorant is a quinacridone, the first solvent can be an acid such as polyphosphoric acid. In addition, when the organic colorant is a phthalocyanine, such as copper phthalocyanine, the first solvent can be sulfuric acid.

The solution of the organic colorant in the first solvent can be prepared by any method known in the art. For example, a colored pigment may be dissolved in the first solvent, with or without the use of heat or shear, which breaks the insoluble pigment down to its constituent organic colorant. Alternatively, the solution may be prepared by forming the organic colorant in the first solvent, resulting in a solution of colorant. For example, as is known in the art, colored pigments, which comprise water-insoluble organic colorants, are prepared by the reaction of various organic colorant precursors, typically as a solution reaction. Thus, the solution of organic colorant may be the product of the reaction of various organic colorant precursors in the first solvent, and can be combined with the aromatic amine, the diazotizing agent, the optional acid and second solvent, and the aqueous medium without isolation of the organic colorant from the first solvent. Since the step of forming the organic colorant as a solution in the first solvent is typically the final synthetic step in the preparation of a colored pigment, the method of the present invention is therefore a method of preparing a modified colored pigment from an organic colorant prior to forming the colored pigment.

An aromatic amine is also used in the method of the present invention, which is an amine compound having the formula H₂N—Ar, wherein Ar is an aromatic group. The aromatic amine further comprises at least one organic group and is therefore a substituted aromatic amine. The organic group can be a non-ionic organic group, which is a group having no apparent charge. Thus, the aromatic amine can be an amine compound having the formula H₂N—Ar—NI, wherein NI is a nonionic group. Examples of non-ionic groups include, but are not limited to, alkyl groups (such as —R″), carboxylic acid esters (such as —COOR″ or —OCOR″), amides (such as —CONHR″, —CONR″₂, —NHCOR″, or —NR″COR″), alkylene oxides, glycols, alcohols, ethers (such as —OR″), ketones (such as —COR″), halogens, and nitriles. In the above formulas, R″ is a branched or unbranched alkyl or alkylene group having 1-20 carbon atoms.

The organic group can also be an ionic organic group, which is a group comprising at least one ionic group, at least one ionizable group, or a mixture of at least one ionic group and at least one ionizable group, and these are preferred for several end-use applications, discussed in more detail below. An ionic group is either anionic or cationic and is associated with a counterion of the opposite charge including inorganic or organic counterions such as Na⁺, K⁺, Li⁺, NH₄⁺, NR′₄⁺ acetate, NO₃⁻, SO₄⁻², R′SO₃⁻, R′OSO₃⁻, OH⁻, and Cl⁻, where R′ represents hydrogen or an organic group such as a substituted or unsubstituted aryl and/or alkyl group. An ionizable group is one that is capable of forming an ionic group in the medium of use. Anionizable groups form anions and cationizable groups form cations. Organic ionic groups include those described in U.S. Pat. No. 5,698,016, the description of which is fully incorporated herein by reference. Thus, the aromatic amine can be an amine compound having the formula H₂N—Ar—I, wherein I is an ionic group, an ionizable group, or a mixture thereof.

The ionic or ionizable group may be an anionic or anionizable group. Anionic groups are negatively charged ionic groups that may be generated from groups having ionizable substituents that can form anions (anionizable groups), such as acidic substituents. They may also be the anion in the salts of ionizable substituents. Representative examples of anionic groups include —COO⁻, —SO₃⁻, —OSO₃⁻, —HPO₃⁻, —OPO₃⁻², and —PO₃⁻². Representative examples of anionizable groups include —COOH, —SO₃H, —PO₃H₂, —R′SH, —R′OH, and —SO₂NHCOR′, where R′ represents hydrogen or an organic group such as a substituted or unsubstituted aryl and/or alkyl group. As specific examples, the aromatic amine comprises at least one sulfonic acid group, carboxylic acid group, or salts thereof.

As another example, the organic group comprises at least one phosphorus-containing group having at least one P—O or P═O bond, such as at least one phosphonic acid group, at least one phosphinic acid group, at least one phosphinous acid group, at least one phosphite group, at least one phosphate, diphosphate, triphosphate, or pyrophosphate groups, partial esters thereof, or salts thereof. Thus, the organic group may comprise at least one phosphonic acid group, partial ester thereof, or salt thereof. Also, the organic group may comprise at least two of these groups, such as at least two phosphonic acid groups, partial esters thereof, or salts thereof. By “partial ester thereof” is meant that the phosphonic acid group may be a partial phosphonic acid ester group having the formula —PO₃RH, or a salt thereof, wherein R is an aryl, alkaryl, aralkyl, or alkyl group. When the organic group comprises at least two phosphonic acid groups or salts thereof, either or both of the phosphonic acid groups may be a partial phosphonic ester group. Also, one of the phosphonic acid groups may be a phosphonic acid ester having the formula —PO₃R₂ while the other phosphonic acid group may be either a partial phosphonic ester group, a phosphonic acid group, or a salt thereof. However, it is preferred that, for this example, at least one of the phosphonic acid groups is either a phosphonic acid, a partial ester thereof, or salts thereof. By “salts thereof” is meant that the phosphonic acid group may be in a partially or fully ionized form having a cationic counterion. When the organic group comprises at least two phosphonic acid groups, either or both of the phosphonic acid groups may be in either a partially or fully ionized form. Thus, the organic group may comprise at least two phosphonic acid groups, wherein either or both may have the formula —PO₃H₂, —PO₃H⁻ M⁺ (monobasic salt), or —PO₃⁻² M⁺₂ (dibasic salt), wherein M⁺ is a cation such as Na⁺, K⁺, Li⁺, or NR₄⁺, wherein R, which can be the same or different, represents hydrogen or an organic group such as a substituted or unsubstituted aryl and/or alkyl group.

More specifically, the organic group may comprise at least one geminal bisphosphonic acid group, partial esters thereof, or salts thereof—that is, the organic group may comprise at least two phosphonic acid groups, partial esters thereof, or salts thereof that are directly bonded to the same carbon atom. Such a group may also be referred to as a 1,1-diphosphonic acid group, partial ester thereof, or salt thereof. Thus, for example, the organic group may comprise a group having the formula —CQ(PO₃H₂)₂, partial esters thereof, or salts thereof. Q is bonded to the geminal position and may be H, R, OR, SR, or NR₂ wherein R, which can be the same or different, is H, a C1-C18 saturated or unsaturated, branched or unbranched alkyl group, a C1-C18 saturated or unsaturated, branched or unbranched acyl group, an aralkyl group, an alkaryl group, or an aryl group. For example, Q may be H, R, OR, SR, or NR₂, wherein R, which can be the same or different, is H, a C1-C6 alkyl group, or an aryl group. Thus, Q can be H, OH, or NH₂. Furthermore, the organic group may comprise a group having the formula —(CH₂)ₙ—CQ(PO₃H₂)₂, partial esters thereof, or salts thereof, wherein Q is as described above and n is 0 to 9, such as 1 to 9, 0 to 3, 1 to 3, 0, or 1.

The ionic or ionizable group may also be a cationic or cationizable group. Cationic groups are positively charged organic ionic groups that may be generated from ionizable substituents that can form cations (cationizable groups), such as protonated amines. For example, alkyl or aryl amines may be protonated in acidic media to form ammonium groups —NR′₂H⁺, where R′ represent an organic group such as a substituted or unsubstituted aryl and/or alkyl group. Cationic groups may also be positively charged organic ionic groups. Examples include quaternary ammonium groups (—NR′₃⁺) and quaternary phosphonium groups (—PR′₃⁺). Here, R′ represents hydrogen or an organic group such as a substituted or unsubstituted aryl and/or alkyl group. As a specific example, the aromatic amine may comprise at least one amine group or a salt thereof, or at least one quaternary ammonium group.

Thus, the aromatic amine may be an amine compound having the formula H₂N—Ar—I or H₂N—Ar—NI. The group Ar preferably represents an arylene or heteroarylene group (such as a phenylene, naphthylene, or biphenylene) or an alkarylene group (such as aminobenzyl amine). The group Ar is directly attached to the amine group and is further substituted with an I group or an NI group, which are both described in more detail above. Specific examples of aromatic amines having the formula H₂N—Ar—I include p-aminobenzoic acid and sulfanilic acid. Specific examples of aromatic amines having the formula H₂N—Ar—NI include aminobenzyl carboxylic acid esters, aminobenzyl carboxylic acid amides.

The aromatic group of the aromatic amine may further be substituted with one or more additional functional groups. Examples of functional groups include, but are not limited to, R″, OR″, COR″, COOR″, OCOR″, carboxylates, halogens, CN, NR″₂, SO₃H, sulfonates, sulfates, NR″(COR″), CONR″₂, NO₂, PO₃H₂, phosphonates, phosphates, N═NR″, SOR″, NSO₂R″, wherein R″, which can be the same or different, is independently hydrogen or a branched or unbranched, substituted or unsubstituted, saturated or unsaturated C₁-C₂₀ hydrocarbon group, e.g., an alkyl, alkenyl, alkynyl, substituted or unsubstituted aryl, substituted or unsubstituted heteroaryl, substituted or unsubstituted alkaryl, or substituted or unsubstituted aralkyl group.

A diazotizing agent is also combined in the method of the present invention. A diazotizing agent is any reagent that reacts with an amine group to form a diazonium salt. Examples include nitrous acid and nitrite salts. Preferably, the diazotizing agent is a salt having a nitrite counterion such as sodium nitrite, potassium nitrite, or calcium nitrite. The diazotizing agent may also be the aromatic amine comprising a cationic group, such as a quaternary ammonium group, in which the counterion of the quaternary ammonium group is a nitrite.

The aqueous medium used in the method of the present invention is any medium containing greater than 50% by weight water, including greater than 60%, greater than 75%, or greater than 95% by weight water. The aqueous medium can also be water. Thus, the aqueous medium can be, for example, water or mixtures of water with water miscible solvents such as alcohols.

In the method of the present invention, an optional second solvent may be used. This may provide, for example, improved solubility of the aromatic amine and/or the diazotizing agent, in the solution of the organic colorant. A variety of different solvents may be used as the second solvent, depending on the solubility properties of the components. Furthermore, the second solvent may be the same as the first solvent. Thus, the second solvent may be a non-aqueous solvent or it may also be an aqueous solvent, such as water.

In addition, an optional acid may be used in the method of the present invention. Any acid may be used, especially water soluble acids such as HCl and nitric acid, but may also be an organic acid, such as methane sulfonic acid. Such an acid may be particularly useful as an activating agent for the reaction of the aromatic amine and the diazotizing agent, when combined to form a diazonium salt. Alternatively, the acid may be used to enhance the solubility of the aromatic amine in the solution of the organic colorant. The acid may be included as a separate component, or it may be added to the aqueous medium. Thus, while the aqueous medium may be any pH, in particular, it can be acidic. Thus, the acid may be added to the aqueous medium in order to form a low pH aqueous medium.

The amounts of each component may be varied in order to obtain a modified colored pigment having desirable overall properties, such as stability in an aqueous dispersion. For example, the organic colorant solution generally contains from about 1 to about 75% by weight organic colorant. The amount of aromatic amine is generally much less than the amount of organic colorant—preferably less than 15% of the molar amount of the organic colorant and more preferably less than 10%, such as between about 1% and 7% of the molar amount of the organic colorant. The diazotizing agent can be used at the same molar amount as the aromatic amine, or in slight excess. When an optional acid is used, the acid is used in an amount known in the art to be sufficient for forming a diazonium salt, which is generally approximately 2 molar equivalents compared to the molar amount of aromatic amine. If the aromatic amine also has an acidic group, less optional acid would be needed. The amount of aqueous solution used is an amount sufficient to cause precipitation of the colorant and is generally dependent on the type of first solvent used. For example, when the first solvent is an acid, such a polyphosphoric acid or sulfuric acid, the aqueous solution can be used in amounts greater than about 10% of the volume of the solution of organic colorant, and, preferably, in large excess of the volume of the organic colorant solution. For example, the ratio of the volume of aqueous solution to the volume of the organic colorant solution can be between about 0.1/1 to about 20/1, including between about 1/1 to about 10/1. Since the aqueous solution can cause precipitation of the organic colorant at levels as low as about 10%, when the optional second solvent is used, generally the amount of second solvent is also low in order to avoid colorant precipitation. Thus, the amount of the second solvent is preferably less than 10% of the volume of the solution of organic colorant. This is particularly true when the second solvent is water and is used to form an aqueous solution of a diazonium salt.

When the solution of the organic colorant in the first solvent, the aromatic amine, the diazotizing agent, the aqueous medium, and the optional acid and second solvent are combined, a modified pigment results. The modified pigment comprises a pigment having attached at least one organic group. The pigment is the pigment that would have resulted from the combination of the organic colorant as a solution in the first solvent and the aqueous medium. The organic group is any of those described above for the aromatic amine. The modified pigment may be in the form of a dispersion, such as an aqueous dispersion, or may be wet solid, such as a presscake, depending on the relative amounts of the components that were combined.

The method of the present invention can further comprise a step of adjusting the particle size of the modified colored pigment. For example, the particle size of the modified colored pigment can be reduced, using procedures known in the art, such as milling (including salt milling). Also, if desired, the particle size of the modified colored pigment can be increased, such as by Oswald ripening, i.e. by heating at 50-200° C. with an appropriate solvent for 0.5-15 hours. The type of particle size adjustment is dependent on the desired size of the resulting pigment, which is dependent on the targeted use of that pigment. Typically, for ink jet ink applications, the resulting modified colored pigment is size-reduced.

This particle size reduction step can be done before or after the combining step, but is after the addition of the aqueous medium. For example, the method of the present invention may comprise the steps of forming a solution of an organic colorant in a first solvent and then combining the solution of the organic colorant and an aqueous medium to form a mixture comprising a colored pigment. This colored pigment is not isolated from the mixture, but instead is used directly to form the modified colored pigment. Thus, this mixture may be combined, in any order, with at least one aromatic amine comprising at least one organic group, at least one diazotizing agent, an aqueous medium, and, optionally at least one acid and/or at least one second solvent to form a mixture comprising the modified colored pigment, which can then be further processed to adjust the pigment particle size, such as by milling to reduce the particle size of the modified pigment. Alternatively, the mixture comprising the colored pigment can be processed to adjust the particle size of the colored pigment, and then combined with the components described above, forming the modified colored pigment. Particle size adjustment may also occur both after forming the mixture comprising the colored pigment and forming the modified colored pigment.

As a specific example of the method of the present invention, a solution of a magenta organic colorant can be prepared by heating 1 part by weight 2,5-bis-(p-tolylamino)terephthalic acid (prepared using procedures known in the art) dissolved in 6 parts by weight polyphosphoric acid (PPA) with stirring for 3 hours at 150° C. The resulting solution of 2,9-dimethylquinacridone in PPA can be cooled to 60° C. and combined with an aqueous solution of 4-sulphophenyldiazonium salt (prepared by combining sulfanilic acid with a stoichiometric amount or slight excess of sodium nitrite and 1.2 equivalents of hydrochloric acid in water) and stirred for 2 hours. The volume of the diazonium salt solution should be less than 10% of the volume of the PPA so that precipitation of the organic colorant is minimized. Alternatively, each of these components can be added to the solution of the organic colorant separately. The amount of sulfanilic acid, and therefore of the resulting diazonium salt, would depend on the desired properties of the final modified pigment and could be, for example, 0.3 moles for 1 mole of 2,5-bis-(p-tolylamino)terephthalic acid. The mixture comprising the modified magenta pigment can then be mixed with a large excess of cold water as the aqueous medium (for example, 10 times the volume of the solution of organic colorant), resulting in precipitation of the modified magenta pigment. Alternatively, the aqueous medium could be added to the solution of 2,9-dimethylquinacridone in PPA, forming a mixture comprising a magenta pigment, and the diazonium salt solution, or the components used to form the diazonium salt, could then be added to this mixture, without isolation or purification of the precipitated magenta pigment.

As an additional example, a solution of a cyan organic colorant, prepared by dissolving 1 part of copper phthalocyanine in 10 parts by weight of 62% sulfuric acid, can be combined with an aqueous solution of 4-carboxyphenyldiazonium salt (prepared by combining p-aminobenzoic acid, PABA, with a stoichiometric amount or slight excess of sodium nitrite in water and 2.2 equivalents of nitric acid) at 50-70° C. for 3 hours, thereby forming a mixture comprising a modified cyan pigment. Alternatively, each of these components can be added to the solution of the organic colorant separately. The amount of PABA, and therefore of the resulting diazonium salt, would depend on the desired properties of the final modified pigment and could be, for example, 0.1 moles for 1 mole copper phthalocyanine. The mixture comprising the modified cyan pigment can then be mixed with 10 parts by weight cold water as the aqueous medium, resulting in precipitation of the modified cyan pigment. Alternatively, the aqueous medium could be added to the solution of copper phthalocyanine in sulfuric acid, forming a mixture comprising a cyan pigment, and the diazonium salt solution, or the components used to form the diazonium salt, could then be added to this mixture, without isolation or purification of the precipitated cyan pigment.

As described above, in one embodiment, the method of the present invention comprises the step of combining a solution of an organic colorant and various specified components to form a modified colored pigment. Thus, the method of the present invention is a method of preparing a modified colored pigment from an organic colorant prior to converting this colorant to a pigment. The resulting modified colored pigment would be expected to differ significantly from a modified colored pigment prepared by modification of a colored pigment in a dispersion since, in this case, only the surface would be modified. The modified colored pigment prepared by the method of the present invention would be expected to be both surface modified as well as bulk modified. Such a pigment would be expected to have a reduced color shift.

In addition, it would not be expected that a modified colored pigment could be formed from a solution of an organic colorant, without formation and isolation/purification of a colored pigment first. In particular, since an organic colorant is typically formed by reaction of organic colorant precursors followed by precipitation with an aqueous medium, it would be expected that the components of the reaction medium used to prepare the organic colorant would interfere with the formation of the modified colored pigment.

Thus, the method of the present invention is surprisingly capable of forming a modified colored pigment, comprising a pigment having attached at least one organic group, and this pigment would be both surface modified as well as bulk modified. Furthermore, it would be expected that the resulting modified colored pigment would be useful in a variety of different applications, including, for example, plastic compositions, aqueous inks, aqueous coatings, rubber compositions, paper compositions and textile compositions. In particular, these pigments may be used in aqueous compositions, including, for example, automotive and industrial coatings, paints, toners, adhesives, latexes, and inks, especially inkjet inks.

The foregoing description of preferred embodiments of the present invention has been presented for the purposes of illustration and description. It is not intended to be exhaustive or to limit the invention to the precise form disclosed. Modifications and variations are possible in light of the above teachings, or may be acquired from practice of the invention. The embodiments were chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. A method of preparing a modified colored pigment comprising a pigment having attached at least one organic group, wherein the method comprises the step of combining, in any order, a) a solution of an organic colorant in a first solvent, b) at least one aromatic amine comprising the organic group, c) at least one diazotizing agent, d) an aqueous medium, e) optionally at least one acid, and f) optionally at least one second solvent, to form the modified colored pigment.

2. The method of claim 1, wherein the organic group comprises at least one ionic group, at least one ionizable group, or a mixture thereof.

3. The method of claim 1, wherein,
a) the aromatic amine, the diazotizing agent, the optional acid, and the optional second solvent are combined to form a diazonium reagent;
b) the diazonium reagent and the solution of the organic colorant in the first solvent are combined to form a mixture, and
c) the mixture is combined with the aqueous medium to form the modified colored pigment.

4. The method of claim 1, wherein,
a) the aromatic amine, the diazotizing agent, the optional acid, the optional second solvent, and the aqueous medium are combined to form an aqueous solution of a diazonium reagent, and
b) the aqueous solution of the diazonium reagent and the solution of the organic colorant in the first solvent are combined to form the modified colored pigment.

5. The method of claim 1, wherein the solution of the organic colorant is prepared by forming the organic colorant as a solution in the first solvent.

6. The method of claim 5, wherein the organic colorant is not isolated from the first solvent prior to combining with the aromatic amine, the diazotizing agent, the optional acid, the optional second solvent, and the aqueous medium.

7. The method of claim 1, wherein the solution of the organic colorant is prepared by dissolving the organic colorant in the first solvent.

8. The method of claim 1, wherein the first solvent is a non-aqueous solvent.

9. The method of claim 1, wherein the first solvent is an acid.

10. The method of claim 1, wherein the organic colorant is a blue colorant, a black colorant, a brown colorant, a cyan colorant, a green colorant, a white colorant, a violet colorant, a magenta colorant, a red colorant, an orange colorant, a yellow colorant, or mixtures thereof.

11. The method of claim 1, wherein the organic colorant is an anthraquinone, a phthalocyanine blue, a phthalocyanine green, a disazo colorant, a monoazo colorant, a pyranthrone, a perylene, a heterocyclic yellow colorant, a quinolonoquinolone, an isoindolone, an indanthrones, a quinacridones, or a (thio)indigoid.

12. The method of claim 1, wherein the organic colorant is an anthraquinone, a phthalocyanine blue, a phthalocyanine green, or a quinacridone.

13. The method of claim 2, wherein the organic group comprises at least one —COO⁻, —SO₃⁻, —OSO₃⁻, —HPO₃⁻, —OPO₃⁻², amine, or ammonium group.

14. The method of claim 2, wherein the organic group comprises at least one sulfonic acid group, carboxylic acid group, or salt thereof.

15. The method of claim 2, wherein the organic group comprises at least two phosphonic acid groups, partial esters thereof, or salts thereof.

16. The method of claim 2, wherein the organic group comprises at least one geminal bisphosphonic acid group, partial esters thereof, or salts thereof.

17. The method of claim 1, wherein the diazotizing agent comprises a nitrite.

18. The method of claim 1, wherein the aqueous medium is water.

19. The method of claim 1, wherein the modified colored pigment is in the form of an aqueous dispersion.

20. The method of claim 19, wherein the aqueous dispersion is an inkjet ink composition.

21. The method of claim 1, wherein the modified colored pigment is in the form of a presscake.

22. The method of claim 1, wherein the modified colored pigment is further processed to achieve a desired particle size.

23. A method of preparing a modified colored pigment comprising a pigment having attached at least one organic group, wherein the method comprises the steps of:
i) forming a solution of an organic colorant in a first solvent;
ii) combining the solution of the organic colorant and an aqueous medium to form a mixture comprising a colored pigment;
iii) combining, in any order, a) the mixture comprising the colored pigment, b) at least one aromatic amine comprising the organic group, c) at least one diazotizing agent, d) an aqueous medium, e) optionally at least one acid, and f) optionally at least one second solvent to form a mixture comprising the modified colored pigment; and
iv) size-reducing the modified colored pigment in the mixture.

24. The method of claim 23, wherein the colored pigment is not isolated form the mixture formed in step ii).

25. A method of preparing a modified colored pigment comprising a pigment having attached at least one organic group, wherein the method comprises the steps of:
i) forming a solution of an organic colorant in a first solvent;
ii) combining the solution of the organic colorant and an aqueous medium to form a mixture comprising a colored pigment;
iii) adjusting the, particle size of the colored pigment in the mixture; and
iv) combining, in any order, a) the colored pigment, b) at least one aromatic amine comprising the organic group, c) at least one diazotizing agent, d) an aqueous medium, e) optionally at least one acid, and f) optionally at least one second solvent to form the modified colored pigment.

26. The method of claim 25, wherein the colored pigment is not isolated from the mixture formed in step ii).

27. The method of claim 25, wherein step iii) is a size-reduction step.

28. The method of claim 25, wherein the modified colored pigment formed in step iv) is in the form of an aqueous dispersion.

29. The method of claim 25, wherein the modified colored pigment formed in step iv) is in the form of a presscake.
